(19) United States
(12) Patent Application Publication
(10) Pub. No.: US 2025/0001120 A1
(43) Pub. Date: Jan. 2, 2025

(54) CLEANABLE AND RE-USEABLE HEAT AND MOISTURE EXCHANGER

(71) Applicant: KONINKLIJKE PHILIPS N.V., EINDHOVEN (NL)

(72) Inventors: HENDRIK KLAAS LOUWSMA, DRACHTSTERCOMPAGNIE (DE); THEODOOR STOLK, LANGEZWAAG (DE)

(21) Appl. No.: 18/756,158

(22) Filed: Jun. 27, 2024

Related U.S. Application Data
(60) Provisional application No. 63/523,678, filed on Jun. 28, 2023.

(51) Int. Cl. A61M 16/10

(52) U.S. Cl. CPC A61M 16/1095

(57) ABSTRACT

An HME element for use in an HME arrangement. The HME element includes a body defining a number of passages therethrough, each passage extending between a first face of the body and a second face of the body opposite the first face. Each passage of the number of passages has a width in the range of 0.3 mm to 2.0 mm. A portion of the body defining each passage has a thickness, normal to the passage, of at least 0.2 mm.

### CROSS-REFERENCE TO RELATED APPLICATIONS

This patent application claims the priority benefit under 35 U.S.C. § 119 (e) of U.S. Provisional Application No. 63/523,678, filed on Jun. 28, 2023, the contents of which are herein incorporated by reference.

### BACKGROUND OF THE INVENTION

### 1. Field of the Invention

The disclosed concept relates generally to heat and moisture exchangers for use in arrangements providing a flow of breathing gas to a patient and, more particularly, to elements for heat and moisture exchangers that are cleanable, re-useable, and customizable. The disclosed concept further relates respiratory interface systems utilizing such heat and moisture exchangers.

### 2. Description of the Related Art

Breathing apparatus are used to provide breathing assistance to patients. Examples of such breathing apparatus are CPAP (continuous positive airway pressure), bi-level and/or autotitration PAP (positive airway pressure) apparatus that provide pressure support to a patient for treating obstructive sleep apnea or other breathing disorders. Other examples of such breathing apparatus are ventilators (such as non-invasive ventilation—NIV, as well as invasive ventilation) that provide assisted breathing or flow therapy.

Often as part of therapies provided by such devices and/or for comfort to the patient it is desirable to humidify the air provided to the patient by the breathing apparatus. Such humidity may be provided actively, e.g., via humidifiers, or passively, e.g., via a heat and moisture exchanger (HME), or a combination thereof. HME's function to scavenge heat and humidity from gases exhaled by a patient in order to provide heat and humidity to new gases being provided to the patient. Presently, HME's are a consumable product-after one (sometimes multiple) day(s) of use the component is disposed and replaced by a new one. In such HME's, the active element (i.e., the element scavenging/transferring heat/humidity from exhaled gases to new gases being provided to the patient) is typically a sponge like material or rolled paper arrangement.

The goal of an HME is to capture the heat and moisture from the patients exhaled air. This heat and moisture are released again upon inhaling the cooler and relatively dry ambient air. Through such device the air inhaled by the patient is humidified and heated by his/her exhaled moisture and heat, respectively.

The use time of an HME is in principle only one day as mucus and other contaminants may collect in the HME which can result in organism growth. Hence the HME is disposed after use.

### SUMMARY OF THE INVENTION

Embodiments of the present invention improve upon existing solutions by providing HME arrangements that utilize durable, cleanable, and reusable HME elements.

As a first aspect of the present invention, an HME element for use in an HME arrangement is provided. The HME element comprises a body defining a number of passages therethrough, each passage extending between a first face of the body and a second face of the body opposite the first face, wherein each passage of the number of passages has a width in the range of 0.3 mm to 2.0 mm, and wherein a portion of the body defining each passage has a thickness, normal to the passage, of at least 0.2 mm.

The width may be in the range of 0.6 mm-0.8 mm.

The thickness, normal to the passage, may be at least 0.4 mm.

The number of passages may comprise a plurality of passages. Each passage of the plurality of passages may extend in a straight line between the first face and the second face, and wherein the plurality of passages are disposed parallel to each other.

The body may comprise a metallic material. The metallic material may comprise aluminum.

As another aspect of the present invention an HME arrangement is provided. The HME arrangement comprises: a housing defining a passage therethrough; and a number of HME elements positioned within the housing, each HME element of the number of HME elements comprising a body defining a number of passages therethrough, each passage extending between a first face of the body and a second face of the body opposite the first face, wherein each passage of the number of passages has a width in the range of 0.3 mm to 2.0 mm, and wherein a portion of the body defining each passage has a thickness, normal to the passage, of at least 0.2 mm.

The width may be in the range of 0.6 mm-0.8 mm.

The thickness, normal to the passage may be at least 0.4 mm.

The housing may comprise an access panel moveable among a first position in which the access panel defines in-part the passage, and a second position in which the access panel exposes an opening through a portion of the housing to the passage, and the number of HME elements may be removable from the housing when the access panel is disposed in the second position.

The number of passages may comprise a plurality of passages. Each passage of the plurality of passages may extend in a straight line between the first face and the second face, and the plurality of passages may be disposed parallel to each other.

The body of each HME element may comprise a metallic material. The metallic material may comprise aluminum.

The number of HME elements may comprise a plurality of HME elements positioned in a series arrangement. The body of each HME element of the plurality of HME elements may be spaced a predetermined distance from at least one other body of another HME element of the plurality of HME elements.

As yet a further aspect of the present invention a respiratory interface system for use in providing a regimen of respiratory therapy to a patient is provided. The respiratory interface system comprises: a pressure generating device structured to generate a flow of positive pressure breathing gas; a patient interface structured to engage an airway of the patient; a delivery conduit arrangement having a first end coupled to the pressure generating device and an opposite second end coupled to the patient interface, the delivery conduit arrangement structured to communicate the flow of positive pressure breathing gas from the pressure generating device to the patient interface such that the delivery conduit arrangement and patient interface define a flow path which is structured to convey the flow of positive pressure breathing gas from the pressure generating device to the patent; and an HME arrangement positioned along the flow path, the HME arrangement comprising: a housing defining a passage therethrough; and a number of HME elements positioned within the housing, each HME element of the number of HME elements comprising a body defining a number of passages therethrough, each passage extending between a first face of the body and a second face of the body opposite the first face, wherein each passage of the number of passages has a width in the range of 0.3 mm to 2.0 mm, and wherein a portion of the body defining each passage has a thickness, normal to the passage, of at least 0.2 mm.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

A full understanding of the invention can be gained from the following description of the preferred embodiments when read in conjunction with the accompanying drawings in which:

FIG. 1 is a respiratory interface system including an HME in accordance with an example embodiment of the present invention;

FIG. 2 is a perspective view of a portion of the system of FIG. 1 showing details of the HME thereof, shown with a portion of the HME removed to show details of the HME element;

FIG. 3 is a perspective view of an HME element of the HME of FIG. 2;

FIG. 4 is an elevation view of an end of the HME element of FIG. 2; and

FIGS. 5 and 6 are perspective view of HMEs in accordance with other example embodiments of the present invention shown with a portion of the respective HME removed to show details of the HME elements thereof.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As used herein, the statement that two or more parts or components “engage” one another shall means that the parts exert a force against one another either directly (i.e., “directly engage”) or through one or more intermediate parts or components. As used herein, the word “unitary” means a component is created as a single piece or unit. That is, a component that includes pieces that are created separately and then coupled together as a unit is not a “unitary” component or body. As used herein, the term “number” shall mean one or an integer greater than one (i.e., a plurality).

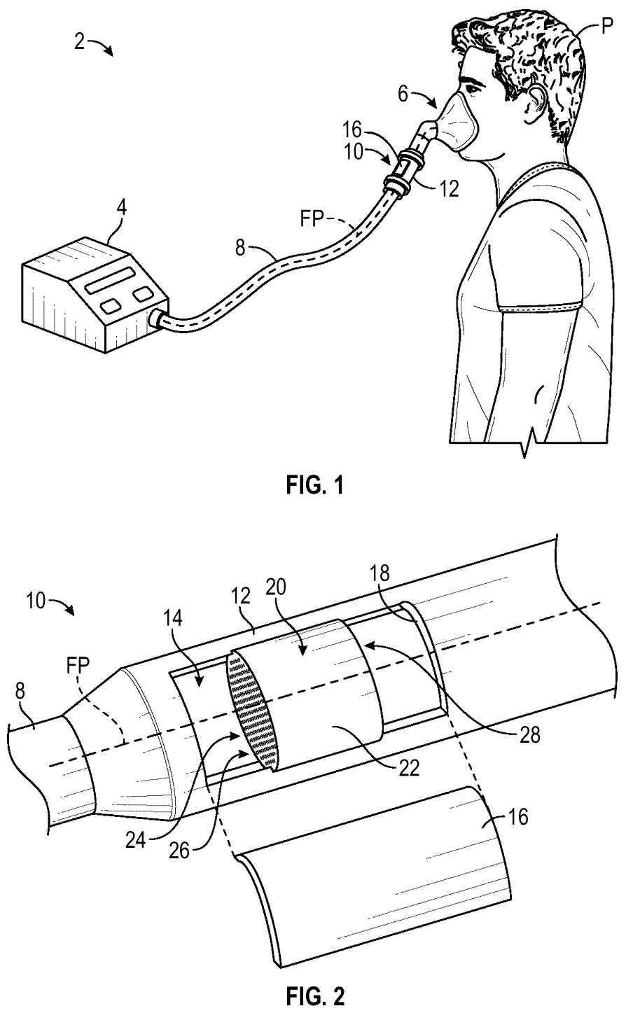

FIG. 1 shows a respiratory interface system 2 for use in providing a regimen of respiratory therapy to a patient P according to one particular, non-limiting example embodiment of the present invention. Respiratory interface system 2 includes a pressure generating device 4, a patient interface 6, and a delivery conduit arrangement 8. Delivery conduit arrangement 8 includes a first end and an opposite second end, with the first end coupled to pressure generating device 4 and the opposite second end being coupled to patient interface 6. Pressure generating device 4 is structured to generate a flow of positive pressure breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, PA), and auto-titration pressure support devices. Delivery conduit arrangement 8 may be formed from one or more (i.e., a number of) conduit sections/couplings (not numbered) and is structured to communicate the flow of positive pressure breathing gas from pressure generating device 4 to patient interface 6. Hence, delivery conduit arrangement 8 and patient interface 6 define a flow path FP (shown in dashed line) from pressure generating device 4 to patent P.

Continuing to FIG. 1 and additionally FIG. 2, respiratory interface system 2 further includes a heat and moisture exchanger (HME) 10 positioned along flow path FP. In the example shown in FIG. 1, HME 10 is positioned immediately adjacent patient interface 6, however, it is to be appreciated that HME 10 may be positioned at other locations along flow path FP (e.g., without limitation, spaced from patient interface 6, provided as a portion of patient interface 6) without varying from the scope of the present invention. HME 10 includes a housing 12 defining a passage 14 therethrough. Housing 12 may be formed from one or more materials the same as/similar to conduit arrangement 8 and/or any other suitable material(s). When HME 10 is positioned within respiratory interface system 2, passage 14 defines a portion of flow path FP. Housing 12 includes an access panel 16 moveable among a first position (e.g., see FIG. 1) in which access panel 16 defines, in-part, passage 14, and a second position (e.g., see FIG. 2) in which access panel 16 exposes an opening 18 through a portion of housing 12 to passage 14.

In the example shown in FIG. 2, access panel 16 is selectively coupleable/uncoupleable (e.g., via a snap fit) from the remainder of housing 12. It is to be appreciated, however, that access panel 16 may interact with the remainder of housing 12 via any suitable arrangement (e.g., without limitation, hinges, sliders, etc.) without varying from the scope of the present invention. It is also to be appreciated that in some examples of the present invention housing 12 does not include access panel 16. In the example shown in FIG. 2, passage 14 has an inner diameter of about 25 mm, as compared to conduit arrangement 8 which typically has an inner diameter in the range of 15 mm-22 mm. It is to be appreciated that the dimensional values provided herein are for exemplary purposes only and thus are not intended to limit the potential scope of the present invention unless otherwise provided (e.g., if particularly provided in one or more claims).

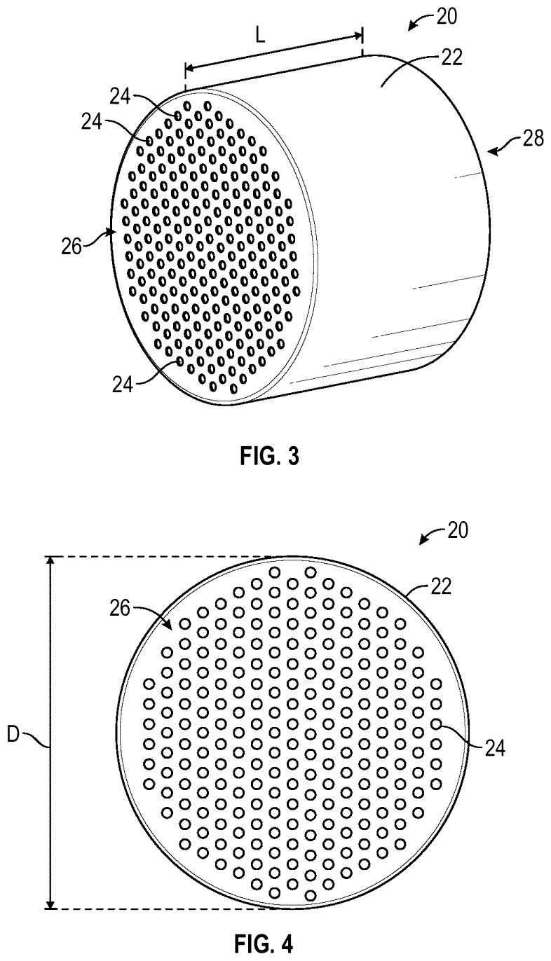

Continuing to refer to FIG. 2, and additionally FIGS. 3 and 4, HME 10 further includes a number of HME elements 20 selectively positioned within housing 12. In the example shown in FIG. 2, HME 10 includes a single HME element 20, however, as discussed further below, more than one HME element 20 may be provided within housing 12 in a series arrangement (i.e., in a line along flow path FP) without varying from the scope of the present invention. Each HME element 20 is removable (e.g., for cleaning) from housing 12 when access panel 16 is disposed/positioned in the second position and thus opening 18 and the number of HME elements 20 are exposed and thus accessible. Each HME element 20 comprises a body 22 defining a plurality of passages 24 extending therethrough between a first face 26 and an opposite second face 28 of body 22. In order to reliably locate body 22 within housing 12/passage 14, housing 12 may define a notched portion (not numbered) extending outward from passage 14 in which an outer portion of body 22 may be disposed/housed when installed within housing 12.

In some example embodiments of the present invention, such as shown in FIGS. 2-4, body 22 is cylindrically shaped and has a length L of about 15 mm and a diameter D of about 25 mm (i.e., generally equal to the inner diameter of passage 14 so as to prevent passage of the flow of positive pressure breathing gas flowing through passage 14 from bypassing passages 24 of body 22). In some example embodiments body 22 is formed from metallic materials, for example, an aluminum material which may be anodized to prevent oxidation from chemicals used in cleaning HME element 20. The use of a solid material ensures a small dead space as compared to foam or paper materials. Further, by using materials with a high heat conductivity, such mass of material provides for improved thermal storage and distribution as compared to materials typically employed in conventional HME's.

In the example embodiment of FIGS. 2-4, passages 24 are straight passages (i.e., passages that extend in a straight line between first face 26 and second face 28) as straight passages are easy and convenient to clean, e.g., by putting HME element 20 in a dishwasher. Passages 24 may be disposed parallel to each other and/or perpendicular to first and second faces 26, 28. In example embodiments of the present invention, passages 24 having a diameter/width in the range of 0.3 mm to 2.0 mm have been employed. Depending on the material of body 22, passages having a diameter/width in the range of 0.6 mm-0.8 mm have been employed. In some example embodiments, all of passages 24 are generally identical, whereas in other example embodiments passages 24 of different diameters have been employed.

In an example embodiment, passages 24 were spaced at least a minimum of 0.2 mm apart, so as to provide a sufficient amount of mass therebetween for absorbing heat from/providing heat to, gases passing therethrough. In other words, in such example the portion of the body defining each passage had a thickness, normal to passage 24, of at least 0.2 mm. In other example embodiments of the present invention passages 24 were spaced between 0.4 mm and 1.0 mm apart. In example embodiments of the present invention as few as one and as many as 500 passages 24 have been employed, however, similar to the other values provided herein, other quantities may be employed without varying from the scope of the present invention. In general, narrower passages in greater quantities are preferred, as such arrangements provide for higher heat capacity of body 22 and more surface area to absorb moisture from exhaled gases from the patient. Higher heat capacity is good for absorbing the heat from the exhaled air. The more heat can be absorbed, the easier the exhaled air is cooled down, which helps the condensation process.

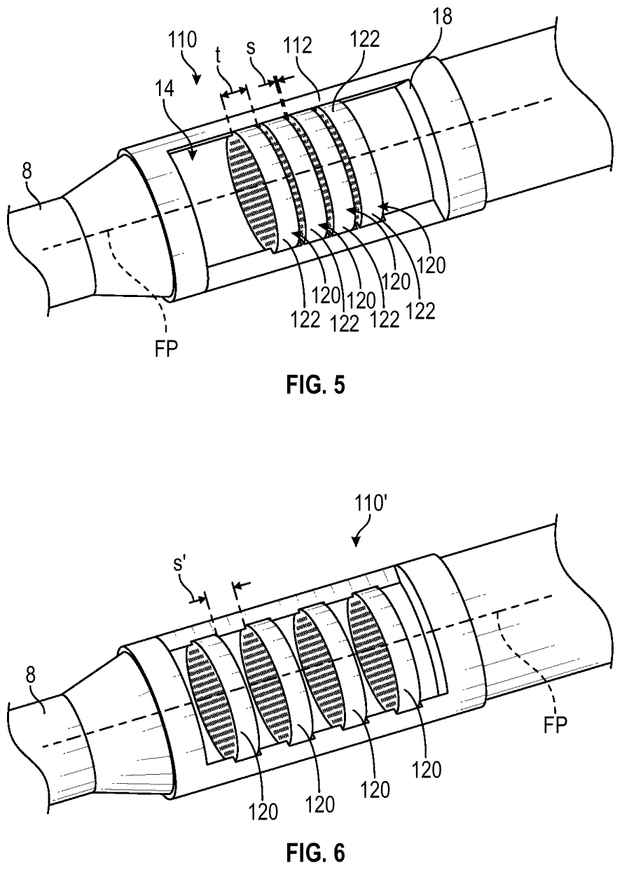

Referring now to FIG. 5, an HME 110 in accordance with another example embodiment of the present invention will now be described. HME 110 is of similar construction and functionality as HME 10 previously discussed in regard to FIGS. 2-4 and thus only differences therefrom will be presently discussed. Unlike HME 10 which utilized a single HME element 20, HME 110 includes a plurality of HME elements 120 positioned within passage 14 of housing 112. Similar to housing 12, housing 112 may define a plurality of notched portions (not numbered) extending outward from passage 14 in which an outer portion of the body 122 of each HME element may be disposed/housed when installed within housing 112. In the example shown in FIG. 5, four HME elements 120 are shown, however it is to be appreciated that other quantities of HME elements 120 may be employed without varying from the scope of the present invention. In such example, body 122 of each HME element 120 has an axial length, or thickness t of 3.6 mm and is spaced from adjacent bodies 122 a spacing s of 1 mm. By utilizing multiple spaced HME elements 120, a proper distribution of a patient's exhalation temperature (around 37° C.) and ambient temperature may be obtained. In contrast, a single HME element averages out such temperatures which is less effective for moisture absorption and release as well as temperature (heat) recycling. Narrow spacing s between HME elements 120 keeps dead space small.

FIG. 6 shows yet a further example embodiment of an HME 110′ that is similar to HME 110 of FIG. 5 except HME elements 120 of HME 110′ are spaced a different spacing s′ from adjacent HME elements 120. In such example, HME elements 120 are spaced a spacing s′ of 4.5 mm.

From the foregoing it is thus to be appreciated that embodiments of the present invention provide HME arrangements for use in system providing regimens of respiratory therapy to patients that utilize durable, cleanable, and reusable HME elements unlike any conventional solutions.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of disclosed concept which is to be given the full breadth of the claims appended and any and all equivalents thereof.

What is claimed is:

1. An HME element for use in an HME arrangement, the HME element comprising a body defining a number of passages therethrough, each passage extending between a first face of the body and a second face of the body opposite the first face,
wherein each passage of the number of passages has a width in the range of 0.3 mm to 2.0 mm, and
wherein a portion of the body defining each passage has a thickness, normal to the passage, of at least 0.2 mm.

2. The HME element of claim 1, wherein the width is in the range of 0.6 mm-0.8 mm.

3. The HME element of claim 1, wherein the thickness, normal to the passage, is at least 0.4 mm.

4. The HME element of claim 1, wherein the number of passages comprises a plurality of passages.

5. The HME element of claim 4, wherein each passage of the plurality of passages extends in a straight line between the first face and the second face, and wherein the plurality of passages are disposed parallel to each other.

6. The HME element of claim 1, wherein the body comprises a metallic material.

7. The HME element of claim 6, wherein the metallic material comprises aluminum.

8. An HME arrangement comprising:
a housing defining a passage therethrough; and
a number of HME elements positioned within the housing, each HME element of the number of HME elements comprising a body defining a number of passages therethrough, each passage extending between a first face of the body and a second face of the body opposite the first face,
wherein each passage of the number of passages has a width in the range of 0.3 mm to 2.0 mm, and
wherein a portion of the body defining each passage has a thickness, normal to the passage, of at least 0.2 mm.

9. The HME arrangement of claim 8, wherein the width is in the range of 0.6 mm-0.8 mm.

10. The HME arrangement of claim 8, wherein the thickness, normal to the passage is at least 0.4 mm.

11. The HME arrangement of claim 8, wherein:
the housing comprises an access panel moveable among a first position in which the access panel defines in-part the passage, and a second position in which the access panel exposes an opening through a portion of the housing to the passage, and
the number of HME elements are removable from the housing when the access panel is disposed in the second position.

12. The HME arrangement of claim 8, wherein the number of passages comprises a plurality of passages.

13. The HME arrangement of claim 12, wherein each passage of the plurality of passages extends in a straight line between the first face and the second face, and wherein the plurality of passages are disposed parallel to each other.

14. The HME arrangement of claim 8, wherein the body of each HME element comprises a metallic material.

15. The HME arrangement of claim 14, wherein the metallic material comprises aluminum.

16. The HME arrangement of claim 8, wherein the number of HME elements comprises a plurality of HME elements positioned in a series arrangement.

17. The HME arrangement of claim 16, wherein the body of each HME element of the plurality of HME elements is spaced a predetermined distance from at least one other body of another HME element of the plurality of HME elements.

18. A respiratory interface system for use in providing a regimen of respiratory therapy to a patient (P), the respiratory interface system comprising:
a pressure generating device structured to generate a flow of positive pressure breathing gas;
a patient interface structured to engage an airway of the patient;
a delivery conduit arrangement having a first end coupled to the pressure generating device and an opposite second end coupled to the patient interface, the delivery conduit arrangement structured to communicate the flow of positive pressure breathing gas from the pressure generating device to the patient interface such that the delivery conduit arrangement and patient interface define a flow path (FP) which is structured to convey the flow of positive pressure breathing gas from the pressure generating device to the patent; and
an HME arrangement positioned along the flow path, the HME arrangement comprising:
a housing defining a passage therethrough; and
a number of HME elements positioned within the housing, each HME element of the number of HME elements comprising a body defining a number of passages therethrough, each passage extending between a first face of the body and a second face of the body opposite the first face,
wherein each passage of the number of passages has a width in the range of 0.3 mm to 2.0 mm, and
wherein a portion of the body defining each passage has a thickness, normal to the passage, of at least 0.2 mm.
